(19) United States
(12) Patent Application Publication
(10) Pub. No.: US 2019/0338239 A1
(43) Pub. Date: Nov. 7, 2019

(54) DRIED MICROORGANISM WITH EXCIPIENT

(71) Applicant: DUPONT NUTRITION BIOSCIENCES APS, Copenhagen K (DK)

(72) Inventors: Christophe Hollard, Madison, WI (US); Geoffrey Babin, Paris (FR)

(21) Appl. No.: 16/479,342

(22) PCT Filed: Jan. 12, 2018
(86) PCT No.: PCT/EP2018/050761

§ 371 Date: Jul. 19, 2019

Related U.S. Application Data
(60) Provisional application No. 62/448,066, filed on Jan. 19, 2017.

(30) Foreign Application Priority Data
Mar. 7, 2017 (EP) 17159494.8

(51) Int. Cl. C12N 1/04; C12N 1/20; A61K 35/742; A61K 35/745; A61K 35/747; A61K 35/66; A61K 9/48; A61K 9/19; A23L 33/135

(52) U.S. Cl. CPC C12N 1/04; C12N 1/20; A61K 35/742; A61K 35/745; A61K 2035/115; A61K 35/66; A61K 9/485; A61K 9/19; A23L 33/135; A61K 35/747

(57) ABSTRACT

The invention provides dried microorganisms with increased shelf-life.

### FIELD OF INVENTION

The present invention relates to the field of dried microorganisms.

### BACKGROUND OF THE INVENTION

It is common to dry microorganisms for storage, for example by freeze- or spray-drying. Such dried microorganisms are used in industrial and food uses, for example in the manufacture of cheese and yoghurt, and also as probiotics. During storage the viability of the dried organism degrades to some extent, so that with time the live cell count diminishes. This is particularly a problem in high-humidity environments.

Probiotics are live microorganisms which are beneficial for human or animal health when administered at appropriate dosages. One way of administering probiotics is through ingestion of dried probiotic mixed with excipients and packaged in capsules or sachets. Unfortunately, dried probiotic cells are not very stable during storage, with the result that live cell counts decrease with time, rendering the treatment less effective. This is particularly true when probiotics are blended with excipients having high humidity content, when container walls are permeable to external atmospheric moisture, and when the probiotic is stored in a high relative humidity environment.

### SUMMARY OF THE INVENTION

In a first aspect, the invention provides a composition comprising a blend of (a) dried microorganism, and (b) a phosphate salt in powder form.

In a second aspect, the invention provides a unit microbial dose, containing a composition comprising a mixture of (a) dried microorganism, and (b) a phosphate salt in powder form.

In a third aspect, the invention provides a process for the preparation of a composition comprising the steps:
(i) providing (a) a dried microorganism, and (b) a phosphate salt in powder form; and
(ii) mixing the dried microorganism, and the phosphate salt in powder form, to provide the composition.

### BRIEF DESCRIPTION OF THE DRAWINGS

FIG. 1 shows the % survival after 3 months' storage in humid conditions (a_{w}=0.4) at 30° C. for different bacterial strains mixed with an excipient according to the invention (“K₂HPO₄”) versus conventional excipient (“MCC”) or no excipient (“Freeze-dried probiotic”).

FIG. 2 shows the % survival after 3 months at 30° C. as a function of a_{w}(a_{w}=0.1, a_{w}=0.2 and a_{w}=0.3), for freeze-dried Lactobacillus acidophilus mixed with excipient according to the invention (“K₂HPO₄”) versus conventional excipient (“MCC”).

FIG. 3 shows the % survival of Lactobacillus acidophilus as a function of the percentage of K₂HPO₄ excipient in a blend under humid conditions (a_{w} 0.4) at 30° C. after 6 months.

FIG. 4 shows the % survival of Lactobacillus acidophilus as a function of the percentage of K₂HPO₄ excipient in a blend with MCC under humid conditions (a_{w} 0.4) at 30° C. after 6 months.

FIG. 5 shows the impact of pH of excipient on the % survival of freeze-dried Lactobacillus acidophilus powder under dry conditions (a_{w} 0.1) at 30° C. after 1 month and after 3 months. “KP” indicates K₂HPO₄ with pH adjusted to the indicated pH, “K₂HPO₄” indicates K₂HPO₄ without pH adjustment, and “MCC” indicates microcrystalline cellulose.

FIG. 6 shows the impact of pH of excipient on the % survival of freeze-dried Lactobacillus acidophilus powder under humid conditions (a_{w} 0.4) at 30° C. after 3 months. “K₂HPO₄” indicates K₂HPO₄ with pH adjusted to the indicated pH, “MCC” indicates microcrystalline cellulose.

### DETAILED DESCRIPTION OF THE INVENTION

All documents referred to herein are incorporated by reference.

The inventors have surprisingly found that the survival of dried microorganisms is improved by using a phosphate salt as excipient. The effect is particularly remarkable in high water activity (a_{w}) environments. A high water activity is considered to be a_{w}>0.15.

Typically, microorganism dry powders, in particular probiotics, are blended with excipients to standardize the microorganism concentration. One of the most used excipients, especially when the microorganism is incorporated in capsules and sachets, is microcrystalline cellulose (MCC). The inventors have used MCC as a reference excipient. The inventors found that the use of phosphate salts can provide for greater than 60% survival of dried microorganism over 3 months compared to almost 0% survival in MCC excipient. This effect is particularly remarkable in high water activity environments (a_{w}>0.15, particularly a_{w}>0.2, more particularly a_{w}>0.3).

Water activity is preferably measured by dew point hygrometer.

Microorganism survival rate is expressed in two different ways.

% Survival=(CFU after storage/CFU t₀)×100% A) Survival percent

Log loss=Log(CFU t₀)−Log(CFU after storage) B) Log loss

### Dried Microorganism

The composition of the present invention contains a dried microorganism. The microorganism, in particular a probiotic, may be dried by any means, however, freeze-drying and spray-drying are preferred, with freeze-drying being particularly preferred.

The expression microorganism is meant to encompass any bacteria or yeast, or mixtures of these, and in particular a probiotic.

The term probiotic includes any live microorganisms which are administered to a host with a view to conferring a health benefit on the host. In particular, it may be a yeast or a bacterium, or mixtures of any of these.

The dried microorganism, in particular a probiotic, may be provided in any form suitable for delivery. For example, the dried microorganism, in particular a probiotic, may be provided in form of granules or powder. In one aspect the microorganism, in particular a probiotic, is in powder form.

The composition of the present invention may contain one species of microorganism, in particular a probiotic, one strain of microorganism, in particular a probiotic, a mixture of species of microorganism, in particular a probiotic, or a mixture of strains of microorganism, in particular a probiotic. In one aspect the composition of the present invention contains one species of microorganism, in particular a probiotic and, optionally, one strain of microorganism, in particular a probiotic. In one aspect the composition of the present invention contains a mixture of strains of microorganism, in particular a probiotic. In one aspect the composition of the present invention contains a mixture of species of microorganisms, in particular probiotics.

In a preferred embodiment, the microorganism, in particular a probiotic, is selected from lactobacilli, bifidobacteria, saccharomyces and mixtures thereof.

In a further preferred embodiment the microorganism, in particular a probiotic, is selected from species selected from Bacillus coagulans, Bifidobacterium longum subsp. infantis, Lactobacillus acidophilus, Lactobacillus paracasei, Lactobacillus johnsonii, Lactobacillus plantarum, Lactobacillus reuteri, Lactobacillus reuteri protectis, Lactobacillus reuteri prodentis, Saccharomyces boulardii, Lactobacillus rhamnosus, Lactobacillus casei, Lactobacillus plantarum, Lactobacillus paracasei and mixtures thereof.

In a further preferred embodiment the microorganism, in particular a probiotic, is selected from species selected from Lactobacillus acidophilus, Lactobacillus casei, Bifidobacterium lactis, and mixtures thereof.

In one aspect the microorganism, in particular a probiotic, is selected from microorganisms of the strains Bacillus coagulans GBI-30, 6086, Bifidobacterium longum subsp. infantis 35624, Lactobacillus acidophilus NCFM, Lactobacillus paracasei St11 (or NCC2461), Lactobacillus johnsonii La1 (=Lactobacillus LC1, Lactobacillus johnsonii NCC533), Lactobacillus plantarum 299v, Lactobacillus reuteri A TCC 55730 (Lactobacillus reuteri SD2112), Lactobacillus reuteri protectis (DSM 17938, daughter strain of A TCC 55730), Lactobacillus reuteri prodentis (DSM 17938/A TCC 55730 and A TCC PTA 5289 in combination), Saccharomyces boulardii, Lactobacillus rhamnosus GR-1, Lactobacillus reuteri RC-14, Lactobacillus acidophilus CL1285, Lactobacillus casei LBC80R, Lactobacillus plantarum HEAL 9, Lactobacillus paracasei 8700:2, and mixtures thereof.

In one aspect the microorganism, in particular a probiotic, is selected from probiotics of the strains Lactobacillus acidophilus (NCFM strain), Lactobacillus Casei (LPC37 strain), Bifidobacterium Lactis (HN0019) and mixtures thereof.

The microorganism, in particular a probiotic, may be present in any suitable amount to deliver the required amount of microorganism, in particular a probiotic. The ‘concentration’ of the microorganism, in particular a probiotic in colony forming units (CFU) of microorganism per gram of the composition may also be selected by one skilled in the art. In one aspect the microorganism, in particular a probiotic is present in an amount of at least 1×10⁸ CFU per gram of the composition. In one aspect the microorganism, in particular a probiotic is present in an amount of at least 1×10⁹ CFU per gram of the composition. In one aspect the microorganism, in particular a probiotic is present in an amount of at least 1×10¹⁰ CFU per gram of the composition. In one aspect the microorganism, in particular a probiotic, is present in an amount of from 1×10⁹ to 5×10⁹ CFU per gram of the composition.

When formulated as a unit probiotic dose, the dose contains any desired amount of probiotic. A typical unit dose will contain 10⁸ to 10¹⁴ CFU per dose, more preferably 10⁹ to 10¹² CFU per dose, particularly preferably 10⁹ to 10¹¹ CFU.

The microorganism, in particular a probiotic is in dried form, preferably spray-dried or freeze-dried, in particular freeze-dried. Preferably the dried microorganism, in particular a probiotic has a water activity of no greater than 0.4, more preferably no greater than 0.3, particularly preferably no greater than 0.2. More particularly preferably, the dried microorganism, in particular a probiotic has a water activity of no greater than 0.1.

### Phosphate Salt

The composition of the present invention contains a phosphate salt. A phosphate salt is any salt of phosphoric acid (H₃PO₄), and includes salts based on dihydrogen phosphate (H₂PO₄⁻), hydrogen phosphate (HPO₄²⁻), and phosphate (PO₄³⁻). In a preferred embodiment the composition of the present invention contains a salt of hydrogen phosphate (HPO₄²⁻), or a mixture of hydrogen phosphate (HPO₄²⁻) and dihydrogen phosphate (H₂PO₄⁻).

Both hydrated and anhydrous phosphate salts can be used.

The cation of the phosphate salt is not particularly limited. In a preferred embodiment, the cation of the phosphate salt is selected from sodium, potassium, calcium, and magnesium. Examples of suitable phosphate salts include K₂HPO₄, KH₂PO₄, Na₂HPO₄, NaH₂PO₄, MgHPO₄, Mg[H₂PO₄]₂, CaHPO₄, Ca[H₂PO₄]₂. In a particularly preferred embodiment, the phosphate salt is a potassium phosphate salt.

Preferably the phosphate salt is dipotassium phosphate (K₂HPO₄), or a mixture of dipotassium phosphate (K₂HPO₄) and potassium dihydrogen phosphate (KH₂PO₄).

In a preferred embodiment, the phosphate salt is pH adjusted to between pH 6 and pH 9, preferably between pH 6.5 and 8, more particularly preferably pH 6.8 to 7.2. By the expression “pH of the salt” is meant the pH of a solution when the salt is dissolved in water. The pH may be adjusted by using blends of dihydrogen phosphate, hydrogen phosphate and phosphate. In a preferred embodiment a blend of HPO₄²⁻ and H₂PO₄⁻ salts is used. The relative amounts of these salts to yield a desired pH is well known. For example, a molar ratio of 61.5/38.5 HPO₄²⁻/H₂PO₄⁻ gives a pH of 7. Alternatively, pH adjusted salts may be prepared by titrating solutions of salts of PO₄³⁻ and/or HPO₄²⁻ with an acid or base to the desired pH and then drying the resulting solution, for example, by spray drying.

The phosphate salt may be present in the composition of the invention in any suitable amount to provide the desired stabilisation of the dried microorganism, in particular a probiotic. In the present context, the wt %'s are given with respect to the total weight of microorganism, in particular a probiotic and phosphate salt. In one aspect, the phosphate salt is present in an amount of at least 10% by weight of the composition. In one aspect, the phosphate salt is present in an amount of at least 20% by weight of the composition. In one aspect, the phosphate salt is present in an amount of at least 30% by weight of the composition. In one aspect, the phosphate salt is present in an amount of at least 40% by weight of the composition. In one aspect, the phosphate salt is present in an amount of at least 50% by weight of the composition. In one aspect, the phosphate salt is present in an amount of at least 60% by weight of the composition. In one aspect, the phosphate salt is present in an amount of at least 70% by weight of the composition. In one aspect, the phosphate salt is present in an amount of at least 80% by weight of the composition.

In one aspect, the phosphate salt is present in an amount of from 10 to 90% by weight of the composition. In one aspect, the phosphate salt is present in an amount of from 20 to 90% by weight of the composition. In one aspect, the phosphate salt is present in an amount of from 30 to 90% by weight of the composition. In one aspect, the phosphate salt is present in an amount of from 40 to 90% by weight of the composition, more preferably 50 to 80% by weight. In one aspect, the phosphate salt is present in an amount of from 50 to 90% by weight of the composition. In one aspect, the phosphate salt is present in an amount of from 60 to 90% by weight of the composition. In one aspect, the phosphate salt is present in an amount of from 70 to 90% by weight of the composition. In one aspect, the phosphate salt is present in an amount of from 80 to 90% by weight of the composition. In the present context, the wt %'s are given with respect to the total weight of microorganism, in particular a probiotic and phosphate salt.

When components other than microorganism, in particular a probiotic, and phosphate salt are present in the composition, the phosphate salt is present in an amount of at least 10% by weight of the total composition. In one aspect, the phosphate salt is present in an amount of at least 20% by weight of the total composition. In one aspect, the phosphate salt is present in an amount of at least 30% by weight of the total composition. In one aspect, the phosphate salt is present in an amount of at least 40% by weight of the total composition. In one aspect, the phosphate salt is present in an amount of at least 50% by weight of the total composition. In one aspect, the phosphate salt is present in an amount of at least 59%, or 60% by weight of the total composition. In one aspect, the phosphate salt is present in an amount of at least 70% by weight of the total composition. In one aspect, the phosphate salt is present in an amount of at least 80% by weight of the total composition.

A suitable mixture is 80 wt % phosphate salt and 20 wt % microorganism, in particular a probiotic.

The phosphate salt is preferably in the form of a powder. Preferably the particle size distribution has a D10 value in microns of 5-120 (more preferably 5-90), a D50 value in microns of 70-180 (more preferably 80-140), and a D90 value in microns of 160-400 (more preferably 180-350).

The survival rate of microorganism, in particular a probiotic is particularly increased under high water activity (a_{w}>0.15, preferably a_{w}>0.2, more preferably a_{w}>0.3) conditions.

The composition of the invention preferably increases the survival of microorganism, in particular a probiotic by at least 30% as compared to microorganism alone, more preferably by at least 40%, particularly preferably more than 60%.

The composition of the invention preferably increases the survival of microorganism, in particular a probiotic under humid conditions (a_{w}>0.15, preferably >0.2, more preferably >0.3) by at least 1% to 30% as compared to microorganism alone, more preferably by at least 20%, particularly preferably more than 30%.

The composition of the invention preferably increases the survival of probiotic by at least 30% as compared to probiotic with MCC as excipient, more preferably by at least 40%, particularly preferably more than 60%.

The composition of the invention preferably increases the survival of probiotic under humid conditions (a_{w}>0.15, preferably >0.2, more preferably >0.3) by at least 30% as compared to probiotic with MCC as excipient, more preferably by at least 40%, particularly preferably more than 60%.

### Additional Components

The composition of the present invention may contain only probiotic and phosphate salt or it may contain one or more additional components.

In one embodiment the composition further comprises additional excipients such as maltodextrin, microcrystalline cellulose (MCC), prebiotics such as inulin, fructooligosaccharides, galactooligosaccharides, polydextrose, flow aid agents such silica, magnesium stearate.

When additional components are present, preferably they constitute less than 20 wt % of the total composition, more preferably less than 10 wt %.

### Use

The composition of the invention may be used for administration to a human or animal as a probiotic, or it may be used for industrial or food applications. Typical food applications include the production of cheese, yoghourt, fermented soy products (such as miso, natto, etc.), sauerkraut, comestible alcohol products, etc. Typical industrial applications include the production of raw or finished materials by fermentation such as industrial alcohol production.

### Forms

The composition of the invention may be in the form of a bulk powder mix, for example for storage or transport before food or industrial use or before administration to a human or animal, and/or before division into suitable dosage forms.

In a further aspect, the invention provides a unit microbial dose for administration to a human or animal. The unit microbial dose comprises a suitable amount of the composition of the invention, which may be packaged, for example, in sachets, capsules or tablets. A typical unit dose will contain 10⁸ to 10¹⁴ CFU per dose, more preferably 10¹⁰ to 10¹² CFU per dose.

### Process

In a further aspect the invention provides a process for the preparation of the composition of the invention comprising (i) providing (a) a dried microorganism, and (b) a phosphate salt in powder form; (ii) mixing the dried microorganism, and the phosphate salt in powder form, to provide the composition. In a preferred embodiment of the process, the microorganism is freeze-dried.

The mixing can be performed by any method that does not damage the microorganism. For example, rotation or shaking in a suitable container, and/or mixing with a mixing implement, such as a paddle.

### EXAMPLES

### Materials

The experiments were conducted with the following freeze-dried probiotics: Lactobacillus acidophilus (NCFM strain), Lactobacillus casei (LPC37 strain), Bifidobacterium lactis (BBi), Bifidobacterium lactis (BBL), and Bifidobacterium lactis (HN0019).

Microcrystalline cellulose was supplied by Mingtai Chemical Company, and the di-potassium phosphate was obtained from BK Glulhi Gmbh Company. Capsules used were Vcaps, size 0, CS, hypromellose from Capsugel cie.

Silica was Sipernat 50s, obtained from Evonik industries AG.

Magnesium stearate was obtained from Aceto corporation.

Maltodextrin (IT6) was obtained from Roquette.

### Analytical Methods

Water Activity Measurement (a_{w})

An Aqualab 3TE, Decagon was used for the measurements of water activity. The sample (about 1 g) is equilibrated within the headspace of a sealed chamber containing a mirror, an optical sensor, an internal fan and infrared temperature sensor.

### Cell Count Method

The cell count method used is the method from quality control laboratory according to the strain. The results were given in colony forming unit per gram of product (CFU/g).

The method consisted of:

(i) 1 g of sample was weighed into a bottle; sterile peptone water was added up to 100 g and the mixture was mixed for 5 minutes at 400 rpm using a bench top shaker. The mixture was left for 20 minutes at room temperature and then mixed again for 5 minutes to obtain a homogenous solution. A 10-2 dilution from the original sample was obtained.

(ii) Subsequent dilutions were carried out at 1:10 steps and were made by adding 1 ml of the solution to 9 ml of peptone water. The solutions were homogenized at each step for 20 seconds using a Vortex system at maximum speed.

(iii) MRS agar and 1% of cysteine was used to plate cells.

(iv) For each determination, 4 plates were counted: two different volumes of cell suspension were plated and each volume was made in duplicate. Then the number of colonies obtained on the plates were added and divided by the total sum of the volumes of cell suspension used for these plates.

(vi) The plates were incubated at 37° C. for 72 hours.

### Example 1

### Preparation of Samples

The various probiotic species were blended with MCC to obtain a CFU between 1.5×10¹⁰ and 3×10¹⁰ CFU/g. The blends were mixed by rotation (about 60 tr/min) in a plastic bottle for 20 min. Then, the capsules were filled with the blends. The preparation of the samples was made in a clean room at 40% RH and 25 deg. C.

### High Humidity Exposure Tests

Maltodextrin was exposed to an atmosphere at 40% RH until equilibrium was reached. As a consequence, the a_{w} of the maltodextrin was close to 0.4. The same method was used to obtain maltodextrin equilibrated at a_{w}'s of 0.3 and 0.1.

Capsules were prepared and filled with:

1. probiotic powder only,

2. probiotic (20 wt %)-MCC (80 wt %) blend, and

3. probiotic (20 wt %)-K₂HPO₄ (80 wt %) blend

These capsules were introduced into a glass bottle. The maltodextrin at an a_{w} of approximately 0.4 was then added on top and the bottle was shaken for the desired time period.

The capsules were stored in an environmental chamber at 30° C. for 6 months. CFU and a_{w} were measured at time 0 months, 1 months, and 3 months to evaluate the impact of excipient type on stability performance.

### Calculation of Survival

Probiotic survival rate was expressed in two different ways.

% Survival=(CFU after storage/CFU t₀)×100% A) Survival percent

Log loss=Log(CFU t₀)−Log(CFU after storage) B) Log loss

### Results

Percent survival of different strains of bacteria in humid conditions (a_{w} 0.4) after three months according to excipient used are shown in Table 1. MCC represents the case where the probiotic was mixed with MCC, K₂HPO₄ represents the case where the probiotics were mixed with K₂HPO₄, and “freeze-dried probiotic” represents the case where the probiotic powder alone was used.

TABLE 1
Percent survival of different bacterial strains after three-months
storage in humid conditions (a_{w} 0.4) and dependence on excipient

| | Lactobacillus | Lactobacillus | Bifidobacterium | Bifidobacterium | Bifidobacterium |
|---|---|---|---|---|---|
| | acidophilus | casei | lactis (BBi) | lactis (BBL) | lactis (HN019) |
| MCC | 0% | 0% | 0% | 1% | 2% |
| K₂HPO₄ | 35% | 1% | 5% | 26% | 20% |
| Freeze-dried | 0% | 0% | 0% | 0% | 0% |
| probiotic | | | | | |

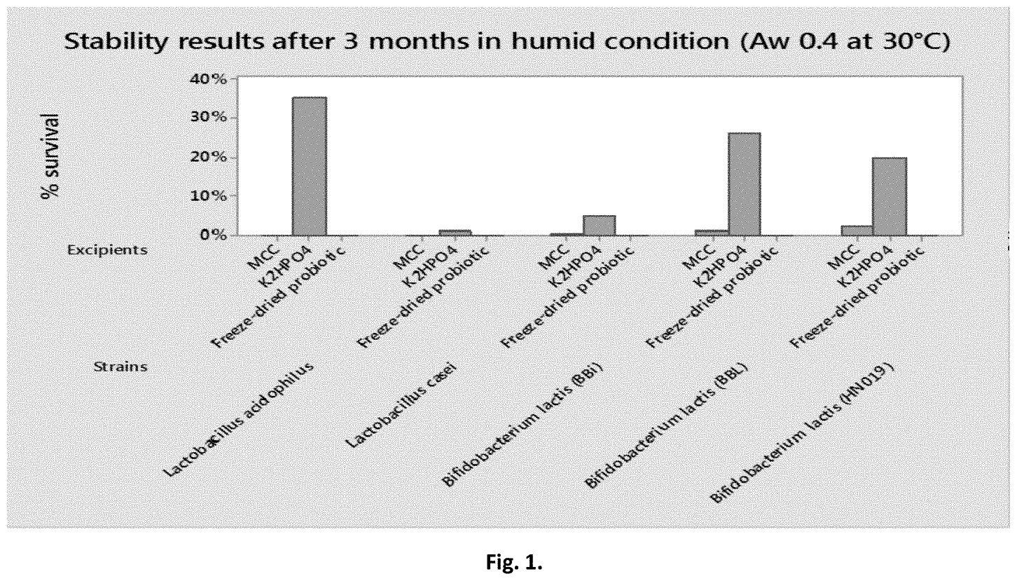

FIG. 1 shows the same results in graphic form. MCC represents the case where the probiotic was mixed with MCC, K₂HPO₄ represents the case where the probiotics were mixed with K₂HPO₄, and “freeze-dried probiotic” represents the case where the probiotic powder alone was used.

Table 1 and FIG. 1 show that the impact of K₂HPO₄ on stability varies as a function of strain. Survival is always higher with K₂HPO₄ as excipient than with MCC or probiotic alone. It is clear that the use of K₂HPO₄ as excipient improves the stability (survival) of the strains, and in particular in humid conditions.

Lactobacillus acidophilus Survival Under Different Humidity Conditions

Percent survival of Lactobacillus acidophilus after three months at 30° C. as a function of a_{w} and excipient is shown in Table 2. MCC represents the case where the probiotic was mixed with MCC, K₂HPO₄ represents the case where the probiotics were mixed with K₂HPO₄.

TABLE 2
Percent survival of Lactobacillus acidophilus after three
months at 30° C. and different a_{w} and dependence on excipient

| | a_{w} | | |
|---|---|---|---|
| | 0.1 | 0.3 | 0.4 |
| MCC | 78% | 6% | 0% |
| K₂HPO₄ | 66% | 39% | 60% |

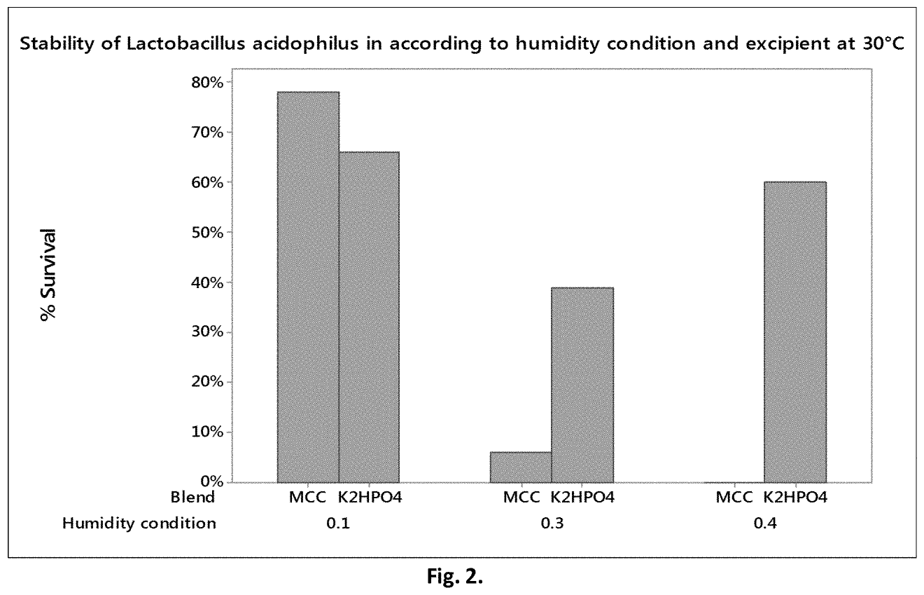

FIG. 2 shows the same results in graphic form. MCC represents the case where the probiotic was mixed with MCC, K₂HPO₄ represents the case where the probiotics were mixed with K₂HPO₄.

Table 2 and FIG. 2 show that the percent survival with K₂HPO₄ are always higher than the percent survival with MCC in the higher humidity conditions (a_{w}>0.1). In the lower humidity condition (a_{w}=0.1), the stabilities with K₂HPO₄ and MCC are similar.

From these results it can be seen that the addition of K₂HPO₄ improves the stability of strains in humid conditions.

### Example 2

This example looks at stability of probiotics in a humid environment as a function of K₂HPO₄ concentration. Sipernat 50s and magnesium stearate were added as flow aids to have a sample composition similar to a commercial blend composition. For this example, freeze-dried Lactobacillus acidophilus powder was used. The example was prepared in two steps. In Part 1, the probiotic was mixed with K₂HPO₄ only. In Part 2, the probiotic was mixed with K₂HPO₄ and MCC.

### Preparation of Samples

Part 1: Probiotics Mixed with K₂HPO₄

Blends consisting of freeze-dried Lactobacillus acidophilus powder, K₂HPO₄, Sipernat 50s, and Magnesium Stearate were prepared. Ten samples were made in which the concentration of freeze-dried Lactobacillus acidophilus was gradually increased and the concentration of K₂HPO₄ gradually decreased while the concentrations of Sipernat 50s and Stearate Magnesium were kept constant. The table below shows the description of the different samples for Part 1.

TABLE 3
Description of different samples for Example 2, Part 1

| | Sample | Sample | Sample | Sample | Sample | Sample | Sample | Sample | Sample | Sample |
|---|---|---|---|---|---|---|---|---|---|---|
| | 1 | 2 | 3 | 4 | 5 | 6 | 7 | 8 | 9 | 10 |
| Lactobacillus | 5 | 10 | 15 | 20 | 25 | 30 | 35 | 40 | 45 | 49.5 |
| Acidophilus (g) | | | | | | | | | | |
| K₂HPO₄ (g) | 44.5 | 39.5 | 34.5 | 29.5 | 24.5 | 19.5 | 14.5 | 9.5 | 4.5 | 0 |
| Sipernat 50s (g) | 0.25 | 0.25 | 0.25 | 0.25 | 0.25 | 0.25 | 0.25 | 0.25 | 0.25 | 0.25 |
| Magnesium | 0.25 | 0.25 | 0.25 | 0.25 | 0.25 | 0.25 | 0.25 | 0.25 | 0.25 | 0.25 |
| stearate (g) | | | | | | | | | | |
| Total mass (g) | 50 | 50 | 50 | 50 | 50 | 50 | 50 | 50 | 50 | 50 |
| Cell count | 3.5E10 | 7E10 | 1.05E11 | 1.4E11 | 1.75E11 | 2.1E11 | 2.45E11 | 2.8E11 | 3.15E11 | 3.47E11 |
| blend (CFU/g) | | | | | | | | | | |
| % K₂HPO₄ | 89% | 79% | 69% | 59% | 49% | 39% | 29% | 19% | 9% | 0% |
| (W/W) | | | | | | | | | | |

Part 2: Probiotics Mixed with K₂HPO₄ and MCC

Blends consisting of freeze-dried Lactobacillus acidophilus powder, and K₂HPO₄, MCC, Sipernat 50s, and Magnesium Stearate were prepared. Nine samples were made in which the concentration of K₂HPO₄ was gradually increased and the concentration of MCC gradually decreased, while the concentrations of the Lactobacillus acidophilus powder, Sipernat 50s and Stearate Magnesium were kept constant. The table below shows the description of the different samples for Part 2.

TABLE 4
Description of different samples for Example 2, Part 2

| | Sample | Sample | Sample | Sample | Sample | Sample | Sample | Sample | Sample |
|---|---|---|---|---|---|---|---|---|---|
| | 11 | 12 | 13 | 14 | 15 | 16 | 17 | 18 | 19 |
| Lactobacillus | 7.5 | 7.5 | 7.5 | 7.5 | 7.5 | 7.5 | 7.5 | 7.5 | 7.5 |
| Acidophilus (g) | | | | | | | | | |
| MCC (g) | 42 | 37 | 32 | 27 | 22 | 17 | 12 | 7 | 0 |
| K₂HPO₄ (g) | 0 | 5 | 10 | 15 | 20 | 25 | 30 | 35 | 42 |
| Sipernat 50s (g) | 0.25 | 0.25 | 0.25 | 0.25 | 0.25 | 0.25 | 0.25 | 0.25 | 0.25 |
| Magnesium | 0.25 | 0.25 | 0.25 | 0.25 | 0.25 | 0.25 | 0.25 | 0.25 | 0.25 |
| stearate (g) | | | | | | | | | |
| Total mass (g) | 50 | 50 | 50 | 50 | 50 | 50 | 50 | 50 | 50 |
| Cell count | 5.25E10 | 5.25E10 | 5.25E10 | 5.25E10 | 5.25E10 | 5.25E10 | 5.25E10 | 5.25E10 | 5.25E10 |
| blend (CFU/g) | | | | | | | | | |
| % K2HPO4 | 0% | 10% | 20% | 30% | 40% | 50% | 60% | 70% | 84% |
| (W/W) | | | | | | | | | |

### Results

### Results of Part 1

Percent survival of Lactobacillus acidophilus as a function of the weight percent of K₂HPO₄ excipient under humid conditions (a_{w} 0.4) after six months at 30° C. is shown in Table 5.

TABLE 5
Percent survival of Lactobacillus acidophilus as
a function of the percent of K₂HPO₄ excipient under humid conditions
(a_{w} 0.4) after six months at 30° C.

| wt % K₂HPO₄ in blend | % Survival |
|---|---|
| 0% | 0% |
| 9% | 0% |
| 19% | 0% |
| 29% | 0% |
| 39% | 0% |
| 49% | 0% |
| 59% | 2% |
| 69% | 9% |
| 79% | 17% |
| 89% | 12% |

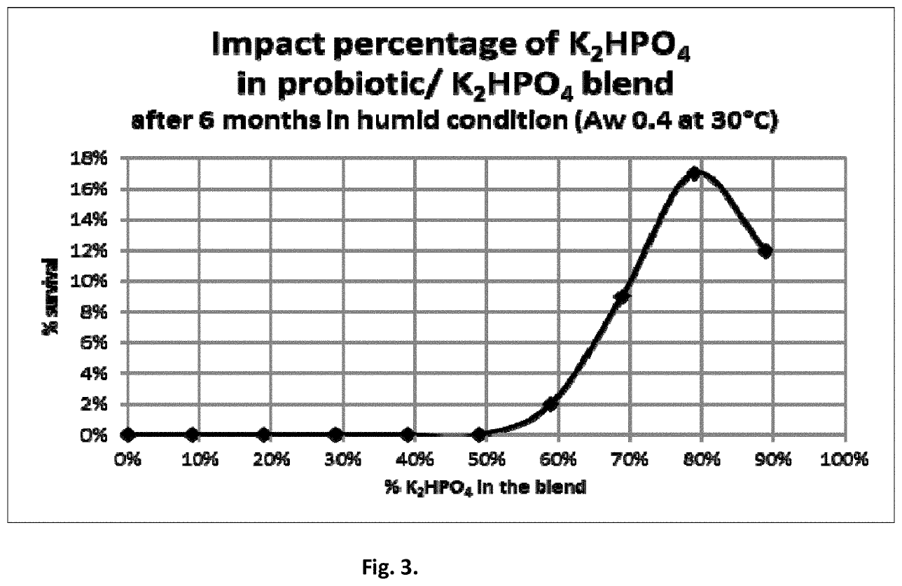

FIG. 3 shows the same results in graphic form.

Table 5 and FIG. 3 show that the survival varies as a function of the amount of K₂HPO₄. The results show that above 50 wt % K₂HPO₄ the survival is significantly increased. Above 80 wt % K₂HPO₄ survival is still significantly better than without K₂HPO₄, although less than at 80 wt %.

### Results of Part 2

Percent survival of Lactobacillus acidophilus as a function of the percentage of K₂HPO₄ excipient in a blend with MCC under humid conditions (a_{w}0.4) at 30° C. after 6 months are shown in Table 6.

TABLE 6
Percent survival of Lactobacillus acidophilus as a function
of the percentage of K₂HPO₄ excipient in a blend with MCC under humid
conditions (a_{w} 0.4) at 30° C. after 6 months

| % K2HPO4 in blend | % Survival |
|---|---|
| 0% | 0.0% |
| 0% | 0.0% |
| 10% | 0.0% |
| 20% | 0.0% |
| 30% | 0.0% |
| 40% | 0.2% |
| 50% | 0.8% |
| 60% | 3.2% |
| 70% | 15.3% |
| 84% | 20.4% |

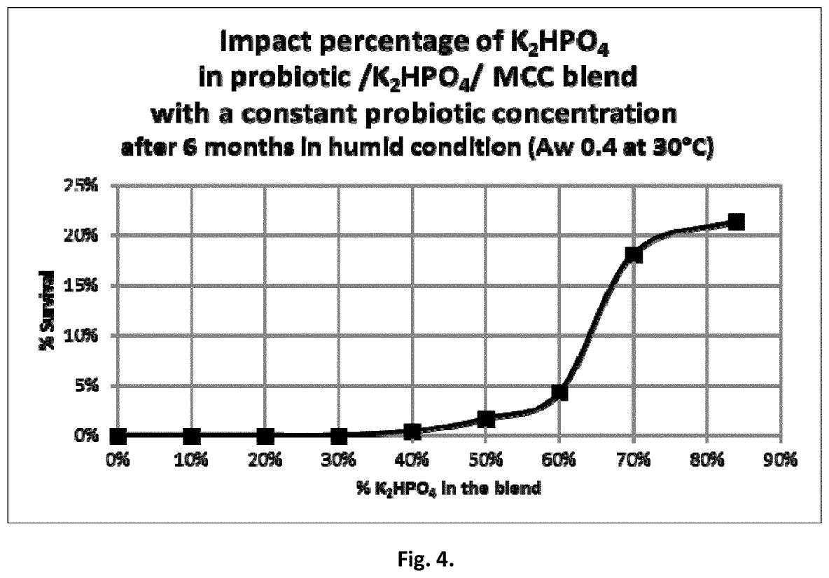

FIG. 4 shows the same results in graphic form.

Table 6 and FIG. 4 show that the survival varies as a function of the K₂HPO₄ percentage, and above 40 wt % K₂HPO₄ is significantly better than without K₂HPO₄.

### Example 3

The effect of pH of K₂HPO₄ excipient on Lactobacillus acidophilus, Bifidobacterium lactis, Lactobacillus casei was evaluated.

Different excipients comprising K₂HPO₄ with various pH values were made (pH 6.5; pH 6.9; pH 7.1; pH 7.6 pH 8.1 and pH 9). The excipients at different pH's were prepared by either of two methods:
1. A solution of K₂HPO₄ was prepared and phosphoric acid added to bring the pH of the solution to the desired value, and the solution was then spray dried to yield a powder; or
2. K₂HPO₄ was blended in dry form with various amounts of KH₂PO₄ calculated to give the desired pH.

### Preparation of Samples

### Part 1: Impact of pH of Excipient on Survival Under Dry Conditions:

Freeze-dried Lactobacillus acidophilus powder was blended with the different excipients at various pH's. The composition of each blend was 80% of excipient K₂HPO₄ powder and 20% freeze-dried probiotic powder. The blends were mixed by rotation (about 60 tr/min) in plastic bottles for 20 min. Sachets were filled with the blends. The preparation of the samples was made in a clean room at 40% RH and 25° C. During testing the sachets were stored at 30° C. in dry humidity (a_{w}≤0.1) for 3 months.

### Part 2: Impact of pH of Excipient on Survival Under Humid Conditions:

Freeze-dried Lactobacillus acidophilus powder was blended with the different K₂HPO₄ powders at different pH's and MCC. The composition of each blend was 80% of excipient and 20% of freeze dry probiotic powder. The blends were mixed by rotation (about 60 tr/min) in plastic bottles for 20 min. The preparation of the samples was made in a clean room at 40% RH and 25° C. Capsules were filled with the blends and the capsules were introduced into a glass bottle. Maltodextrin at an a_{w} of approximately 0.4 was then added on top and the bottle was shaken. The bottles were stored at 30° C. for 3 months.

A control with 80% of dry MCC and 20% of freeze-dried probiotic powder was prepared and tested in the same way.

### Results

### Results of Part 1

Survival of freeze-dried Lactobacillus acidophilus as a function of pH of K₂HPO₄ excipient after storage under dry conditions (a_{w}≤0.1) at 30° C. for one and three months is shown in Table 7.

TABLE 7
Survival of freeze-dried Lactobacillus acidophilus as a
function of pH of K₂HPO₄ excipient after storage under dry conditions
(a_{w} ≤ 0.1) at 30° C. for one and three months

| Excipient | % Survival after 1 month | % Survival after 3 months |
|---|---|---|
| K₂HPO₄ pH 6.5 | 63% | 59% |
| K₂HPO₄ pH 6.9 | 87% | 88% |
| K₂HPO₄ pH 7.1 | 73% | 63% |
| K₂HPO₄ pH 7.6 | 68% | 49% |
| K₂HPO₄ pH 8.1 | 71% | 68% |
| K₂HPO₄ (pH | 58% | 59% |
| unadjusted) | | |
| MCC | 50% | 12% |

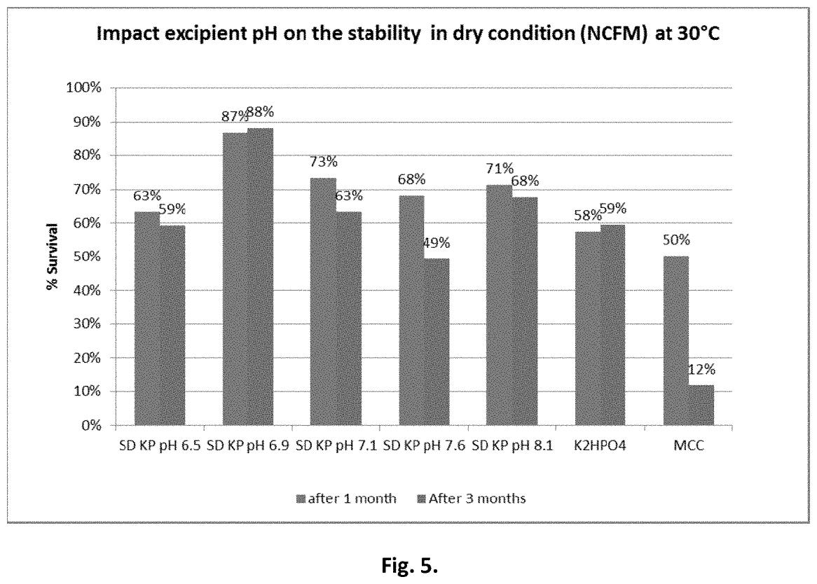

FIG. 5 shows the same results in graphic form.

Table 7 and FIG. 5 show that survival/stability under dry conditions varies as a function of pH, but in all cases is better than survival with MCC as sole excipient. Stability was impacted by the pH of excipient. For stability under dry conditions, the optimal pH of K₂HPO₄ is between 6.9 and 7.1. Other species evaluated included Bifidobacterium lactis and Lactobacillus casei.

### Results of Part 2

Survival of freeze-dried Lactobacillus acidophilus as a function of pH of K₂HPO₄ excipient after storage under humid conditions (a_{w}=0.4) at 30° C. for three months are shown in Table 8.

TABLE 8
Survival of freeze-dried Lactobacillus acidophilus as
a function of pH of K₂HPO₄ excipient after storage under humid
conditions (a_{w} = 0.4) at 30° C. for three months

| Excipient | % Survival |
|---|---|
| K₂HPO₄ pH 6.5 | 37% |
| K₂HPO₄ pH 6.9 | 50% |
| K₂HPO₄ pH 7.1 | 13% |
| K₂HPO₄ pH 7.6 | 14% |
| K₂HPO₄ pH 8.1 | 26% |
| K₂HPO₄ pH 9 | 41% |
| MCC | 1% |

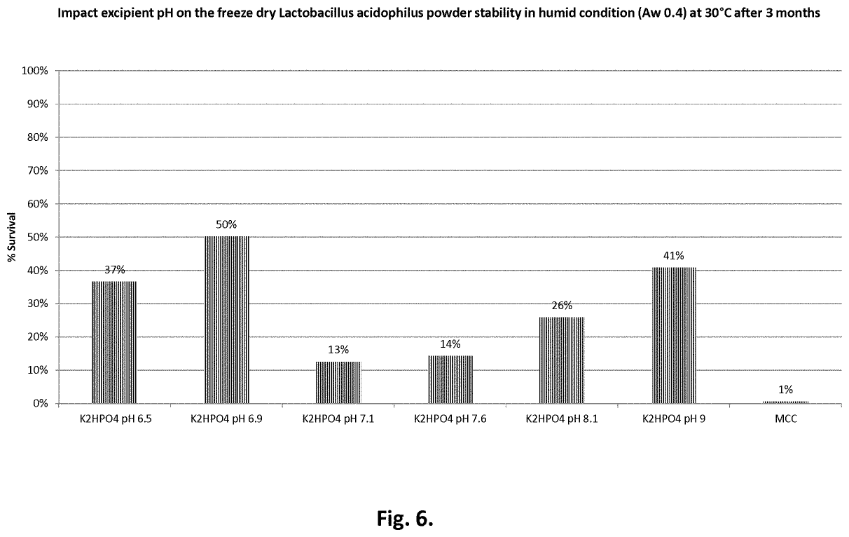

FIG. 6 shows the same results in graphic form.

Table 8 and FIG. 6 show that the stability varies as function of pH value. All excipients with pH adjusted have a better stability than MCC powder under humid conditions. To obtain the best stability under humid conditions, the optimal pH is 6.9.

1. A composition comprising a blend of (a) dried microorganism, and (b) a phosphate salt in powder form.

2. The composition of claim 1, wherein the phosphate is selected from K₂HPO₄, KH₂PO₄, Na₂HPO₄, NaH₂PO₄, MgHPO₄, Mg[H₂PO₄]₂, CaHPO₄, Ca[H₂PO₄]₂.

3. The composition of claim 1, wherein the phosphate salt is selected from K₂HPO₄, KH₂PO₄, Na₂HPO₄, and NaH₂PO₄.

4. The composition of claim 1, wherein the composition comprises a mixture of phosphate salts of HPO₄²⁻ and H₂PO₄⁻.

5. The composition of claim 1, wherein the phosphate salt is pH adjusted to between pH 6 and 9.

6. The composition of claim 1, wherein the phosphate salt is pH adjusted to between 6.8-7.2.

7. The composition of claim 1, wherein the phosphate salt is present in an amount of at least 50 wt % based on the total weight of microorganism and phosphate salt.

8. The composition of claim 1, wherein the phosphate salt is present in an amount of at least 60 wt % based on the total weight of microorganism and phosphate salt.

9. The composition of claim 1, wherein the phosphate salt is present in an amount of at least 80 wt % based on the total weight of microorganism and phosphate salt.

10. The composition of claim 1, wherein the microorganism is freeze-dried.

11. The composition of claim 1, wherein the microorganism is a probiotic.

12. The composition of claim 1, wherein the microorganism is selected from lactobacilli, bifidobacteria, and saccharomyces.

13. The composition of claim 1, wherein the microorganism is selected from Bacillus coagulans, Bifidobacterium longum subsp. infantis, Lactobacillus acidophilus, Lactobacillus paracasei, Lactobacillus johnsonii, Lactobacillus plantarum, Lactobacillus reuteri, Lactobacillus reuteri protectis, Lactobacillus reuteri prodentis, Saccharomyces boulardii, Lactobacillus rhamnosus, Lactobacillus casei, Lactobacillus plantarum, and Lactobacillus paracasei.

14. The composition of claim 1, wherein the microorganism is a Lactobacillus acidophilus.

15. A unit microbial dose comprising a composition according to claim 1.

16. The unit microbial dose of claim 15, in the form of a capsule containing the composition.

17. The unit microbial dose of claim 15, in the form of a sachet containing the composition.

18. The unit probiotic dose of claim 15, in the form of a tablet.

19. The unit probiotic dose of claim 15, in the form of a powdered nutritional formula.

20. A process for the preparation of the composition of claim 1, comprising the steps of:
(i) providing (a) a dried microorganism, and (b) a phosphate salt in powder form; and
(ii) mixing the dried microorganism, and the phosphate salt in powder form, to provide the composition.
